(19) United States
(12) Patent Application Publication
(10) Pub. No.: US 2005/0136017 A1
(43) Pub. Date: Jun. 23, 2005

(54) Hair relaxing composition comprising at least one non-hydroxide imine

(76) Inventors: Gerard Malle, Villiers S/Morin (FR); Damien Lavergne, Paris (FR); Michel Philippe, Wissous (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/988,782

(22) Filed: Nov. 16, 2004

Related U.S. Application Data
(60) Provisional application No. 60/562,218, filed on Apr. 15, 2004.

(30) Foreign Application Priority Data
Nov. 18, 2003 (FR) 0350851

(51) Int. Cl. A61K 7/09

(52) U.S. Cl. 424/70.2

(57) ABSTRACT

The disclosure provides ready-to-use cosmetic compositions for relaxing keratin fibers in a cosmetically acceptable medium free of polyhydroxylated alkane, containing, as a relaxing agent, an imine not belonging to the hydroxide family. The disclosure also provides kits containing compartments to be placed in contact to form the ready-to-use compositions, and processes using these compositions.

This application claims the benefit of U.S. Provisional Application No. 60/562,218, filed Apr. 15, 2004, hereby incorporated by reference.

The present disclosure relates to ready-to-use cosmetic compositions for relaxing keratin fibers, comprising, as a relaxing agent, an imine not belonging to the hydroxide family. The present disclosure also relates to kits comprising compartments to be placed in contact to form the ready-to-use compositions, and also to processes using the compositions.

The term “keratin fibers,” as used herein, encompasses keratin materials, such as fibers of human or animal origin such as head hair, other body hairs, eyelashes, wool, angora, cashmere, fur, and the like. Throughout the specification reference is made to head hair, although the disclosure is not limited to particular keratin fibers.

The terms “relaxing” and “straightening,” as used herein, includes the relaxing, smoothing-out, and decurling of keratin fibers, for example, Caucasian, Asiatic, North African, and African hair.

The term “imine not belonging to the hydroxide family,” as used herein, encompasses any organic compound:
having at least one carbon atom double-bonded to a nitrogen atom and single-bonded to another nitrogen atom and single-bonded to another carbon atom or to a third nitrogen atom;
containing no hydroxide groups in its chemical formula; and
capable of accepting a proton.
   The term also includes organic and mineral salts of such compounds. These “imines not belonging to the hydroxide family” include guanidines and amidines.

The term “polyhydroxylated alkane,” as used herein, means C₁ to C₂₀ alkanes having from 2 to 15 hydroxyl groups.

The expression “between x % and y %,” as used herein, means ranging from x to y %, the limits x and y being included.

The expression “between A and B,” as used herein, means ranging from A to B, the limits A and B being included.

Two techniques may be used to permanently reshape the hair. They are based on breaking the cystine disulfide bonds present in keratin. It will be understood that these techniques can be successfully employed, even if not all of the cystine disulfide bonds are broken.

The first technique comprises opening the disulfide bonds using a composition comprising a reducing agent; optionally rinsing the hair; placing the hair under tension with curlers or the like, or shaping or smoothing out by other means, such as smoothing-out the hair with a large-toothed comb, with the back of a comb or by hand; and reconstituting the disulfide bonds by applying an oxidizing composition (also known as a fixing composition) to the hair, so as to give the hair the desired shape. This technique makes it possible either to make the hair wavy or to relax it, decurl it, or straighten it out.

The second technique comprises performing a lanthionization reaction, using a composition comprising a base belonging to the hydroxide family. This leads to replacement of disulfide bonds (—CH₂—S—S—CH₂—) with lanthionine bonds (—CH₂—S—CH₂—). The lanthionization reaction involves two consecutive chemical reactions:
The first reaction comprises a beta-elimination on the cystine brought about by a hydroxide ion, leading to the breaking of the bond and the formation of dehydroalanine. 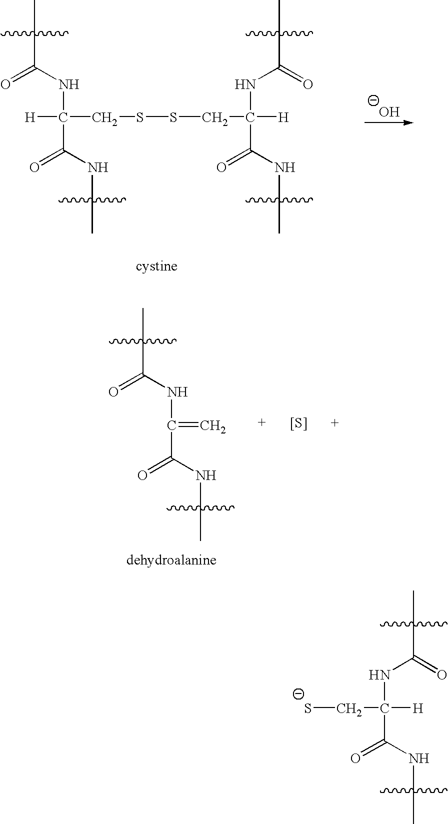
The second reaction comprises a reaction of the dehydroalanine with a thiol group. The double bond of the dehydroalanine formed is a reactive double bond that can react with the thiol group of the cysteine residue that has been released to form a new bond, referred to as a lanthionine bridge or bond or residue.

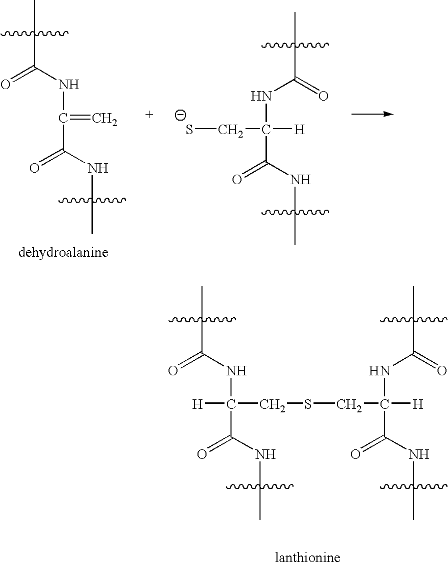

Relative to the first technique using a reducing agent, this lanthionization technique does not require a fixing step, since the formation of the lanthionine bridges is irreversible. It therefore takes place in a single step and makes it possible either to make the hair wavy, relax it, decurl it, or straighten it out. However, it is mainly used to relax naturally frizzy or curly hair.

For the first technique, the reducing compositions generally used for the first step of a permanent-waving or relaxing operation contain thiols, sulfites or bisulfites as reducing agents. These agents are generally used in essentially aqueous medium at concentrations of between 0.5 and 1M to obtain good opening of the disulfide bonds. Thiols that may be used include, but are not limited to, thioglycolic acid, cysteamine, glyceryl monothioglycolate, thiolactic acid, and cysteine. Thioglycolic acid, for example, is efficient at reducing keratin disulfide bonds at alkaline pH, such as in the form of ammonium thioglycolate, and is the product most commonly used in permanent-waving (hair waving). However, it has been found that thioglycolic acid must be used in a sufficiently basic medium (in practice at a pH of between 8.5 and 9.5) if it is desired to obtain curling or relaxing of sufficient intensity. In addition to the drawback of releasing an unpleasant odor requiring the use of fragrances to mask the odor, the use of a thiol at alkaline pH also may lead to degradation of the fibers and to loss of any artificial coloration.

Sulfites and bisulfites are also used for relaxing. They have drawbacks similar to those of thiols, and may have lower efficacy.

Thiols, sulfites, and bisulfites also may have the drawback of having poor stability in aqueous solution.

In general, the durability of the reshaping effects obtained with thiols and sulfites by reduction of disulfides followed by fixing is considered inferior to the effects obtained via the lanthionization technique.

The compositions generally used to perform the lanthionization technique contain as base a hydroxide such as sodium hydroxide, guanidinium hydroxide, and lithium hydroxide. These lanthionization active agents, which make it possible to open disulfide bonds via a beta-elimination reaction, are generally used in a water-in-oil emulsion at concentrations of between 0.4 and 0.6M, by leaving them to act generally for 10 to 15 minutes at room temperature. Sodium hydroxide is the agent most commonly used. Guanidinium hydroxide is also commonly used. Sodium hydroxide and guanidinium hydroxide are the two most common agents used for relaxing or decurling naturally frizzy or curly hair. They may have several advantages over ammonium thioglycolate and sulfites, including the absence of an unpleasant odor, the fact that only one implementation step is required resulting in a shorter treatment time, and much greater durability and efficacy of the reshaping of the hair.

However, these hydroxides have a major drawback of being caustic. This causticity affects the scalp by causing irritation that may be severe. This may be partially overcome by the prior application to the scalp of a greasy protective cream often referred to as “base” or “base cream,” the word “base” used here not having the meaning of a basic agent in the chemical sense. When the protective cream is combined with the hydroxide in a single composition, this is generally referred to as a “no-base” composition. This “no-base” technology is more convenient.

The causticity of the hydroxides also affects the state of the hair by giving it a coarse feel and making it much more fragile. The fragility may make the hair fray, break, or even dissolve if the treatment is prolonged. In some cases, hydroxides also cause decoloration of the natural color of the hair.

Formulations containing sodium hydroxide are generally referred to as “lye relaxers” and those not containing it are generally referred to as “no-lye relaxers.”

The “no-lye” relaxing formulations commonly use guanidinium hydroxide. Since guanidinium hydroxide is unstable, it is generated extemporaneously by mixing guanidine carbonate and a source of very sparingly soluble hydroxide such as calcium hydroxide. The reaction between these two compounds leads to the formation of guanidinium hydroxide and calcium carbonate, which precipitates in the composition. The presence of this precipitate makes the final rinsing of the hair much more difficult and leaves mineral particles on the hair and scalp that give the hair a coarse feel and an unaesthetic appearance resembling dandruff. The recent preference for guanidinium hydroxide (“no-lye”) over sodium hydroxide (“lye”) relaxing formulations appears to arise from better relaxing efficacy and better skin tolerance. However, these technologies using bases of the hydroxide family are still very aggressive treatments to the hair and scalp and require very strict control of the application time to avoid excessive irritation and impairment of the hair that may go as far as breaking. The aggressiveness arising from the causticity of hydroxides is just reason for these compositions for the lanthionization of the hair not to be used for permanent-waving (hair waving), but reserved for relaxing (hair straightening or hair relaxing).

Furthermore, hydroxides are known to be good agents for hydrolyzing amide functions (see for example, March's Advanced Organic Chemistry, 5th Edition, Wiley lnterscience, New York, “Hydrolysis of Amides,” pages 474 et seq.), which thus lead to the breaking of peptide bonds by direct nucleophilic attack. The impairments observed in the case of the hair and keratin materials in the broad sense may be largely due to partial hydrolysis of the amide bonds of keratin.

In light of the above problems, there remains a real need for relaxing compositions that are less aggressive to the hair and the skin, including the scalp.

Various studies have been performed to attempt to simultaneously overcome the drawbacks of reducing agents (used in the first technique) and/or of hydroxides (used in the second technique).

Many reducing agents have been proposed to replace thioglycolic acid, but thioglycolic acid in its ammonium thioglycolate form remains both the reference compound and the compound most widely used in cosmetic formulations, both for shaping and for relaxing and straightening the hair.

U.S. Pat. No. 4,530,830 proposes using a composition based on quaternary ammonium hydroxides to replace sodium hydroxide or guanidinium hydroxide and to improve the skin tolerance. However, these compositions are not entirely satisfactory, either in terms of relaxing or in cosmetic terms.

More generally, many publications describe the combined use of hydroxides, acting as lanthionization active agents, with certain additives generally acting to protect the hair.

Specifically, without using novel lanthionization active agents, the improvements proposed relate mainly to the use of additives to reduce the damage caused to the hair by hydroxides, for example, in the following applications and patents:
PCT Patent Application WO 2002/003937, which describes a composition containing C₃-C₅ monosaccharides,
PCT Patent Application WO 2001/064171, which describes a composition containing complexing agents,
U.S. Pat. No. 5,641,477, which describes a composition containing a hydrogenated starch hydrolysate,
PCT Patent Application WO 02/085317, which describes a composition containing organic nucleophiles, which react during the second step with the dehydroalanine formed with hydroxides, to give new bridges, and
U.S. Pat. No. 5,679,327, which describes a relaxing composition necessarily containing three active constituents for relaxing, namely, an alkaline hydroxide, an alkaline-earth metal hydroxide and a nitrogenous organic base, each of the constituents being present in the composition in a proportion that would be insufficient to effect relaxing if it were used at this same concentration without the other two active agents. In other words, synergism between the three constituents is described, which results in relaxing of the keratin fibers.

Although all these proposals lead to some improvements, they do not make it possible to sufficiently reduce the damage caused by the actual causticity of the hydroxides. In relation to the use of hydroxides for relaxing, U.S. Pat. No. 4,524,787 has also disclosed a ready-to-use composition made from a virtually anhydrous “activating” part comprising an organic base in a solvent such as a polyhydroxylated alkane, and an aqueous part having at least 20% water. However, the latter type of composition is still not satisfactory, either in terms of quality of relaxing or in terms of the resulting mechanical and cosmetic properties of hair that has undergone this treatment.

As indicated previously, the use of reducing agents leads to mediocre durability of the relaxing or decurling and the use of hydroxides, because of their causticity, and limits their use in the relaxing field.

After extensive studies, it has now been discovered, entirely surprisingly and unexpectedly, that the first step of the lanthionization process can be performed with imines not belonging to the hydroxide family. Excellent results in terms of relaxing and cosmetic and mechanical properties of the hair are thus obtained.

Thus, in one aspect, the present disclosure provides cosmetic compositions comprising, in a cosmetically acceptable medium free of polyhydroxylated alkane, at least one imine not belonging to the hydroxide family, wherein the imine reacts with the cystines of keratin fibers, via a beta-elimination reaction, producing dehydroalanine and leading to the formation of lanthionine, to relax the keratin fibers in less than 60 minutes.

The relaxing time may be less than 40 minutes, such as less than 30 minutes.

Imines that may be used include guanidines and amidines, such as, but not limited to:

(1) Guanidine-Type Imines
(a) such as:
   N,N-dimethylguanidine
   N,N′,N″-trimethylguanidine
   N-methylguanidine
   N,N-diethylguanidine
   N-ethylguanidine
   N,N-dimethylimidodicarbonimidic diamide
   N-(tert-butyl)-N″-methylguanidine
   N-(4-aminobutyl)guanidine
   1,3,4,6,7,8-hexahydro-2H-pyrimido[1,2-a]pyrimidine
   N,N″-diisopropylguanidine
   1-methyl-1,3,4,6,7,8-hexahydro-2H-pyrimido[1,2-a]pyrimidine
   N″-(tert-butyl)-N,N,N′,N′-tetramethylguanidine
   N,N,N′,N′-tetraethylguanidine
   N,N″-di-tert-butylguanidine
   N″-[amino(imino)methyl]-N-phenylguanidine
   N,N′,N″-triisopropylguanidine
   pyrrolidine-1-carboximidamide
   N-phenylguanidine
   N-(tert-butyl)-N′,N″-dimethylguanidine
   N-(4-{[amino(imino)methyl]amino}butyl)guanidine
   N″-(tert-butyl)-N-methyl-N′-phenylguanidine
   N,N′,N″-tricyclohexylguanidine
   N-{3-[(4-{[amino(imino)methyl]amino}butyl)amino]propyl}guanidine
   N-(2-hydroxyethyl)-N-methylguanidine
   N-(10-{[amino(imino)methyl]amino}decyl)guanidine
   hydrazinecarboximidamide
   N-hydroxyguanidine
   hydrazinecarboximidohydrazide
   {[amino(imino)methyl]amino}acetic acid
   N-[amino(imino)methyl]thiourea
   N-(1,3-dimethyl-1,3-dihydro-2H-imidazol-2-ylidene)-N-methylamine
   triazane-2-carboximidamide
   morpholine-4-carboximidamide
   N-nitroimidazolidin-2-imine
   [[amino(imino)methyl](methyl)amino]acetic acid
   3-{[amino(imino)methyl]amino}propanoic acid
   (2S)-2-{[amino(imino)methyl]amino}propanoic acid
   5-methoxy-1,2,4-thiadiazol-3-amine
   3-amino-1,2,4-thiadiazole-5-thiol
   N-(3-chloropropyl)guanidine
   N″-cyclopentylidenecarbonohydrazonic diamide
   (2-iminoimidazolidin-1-yl)acetic acid
   morpholine-4-carboximidohydrazide
   4-{[amino(imino)methyl]amino}butanoic acid
   3-[[amino(imino)methyl](methyl)amino]propanoic acid
   3-{[imino(methylamino)methyl]amino}propanoic acid
   N-hydroxymorpholine-4-carboximidamide
   {2-[amino(imino)methyl]hydrazino}(oxo)acetic acid
   1H-pyrazole-1-carboximidamide
   5-(methylsulfanyl)-1,2,4-thiadiazol-3-amine
   1H-1,2,4-triazole-1-carboximidamide
   N-(2-sulfanylethyl)guanidine
   N-(4-methyl-1,3-thiazol-2-yl)guanidine
   2-amino-6-sulfanylpyrimidine-4(3H)-thione
   N-(4-oxo-1,3-thiazolidin-2-yl)guanidine
   6-amino-4-imino-4H-1,3,5-thiadiazine-2-thiol
   2,5-dimethylpyrazolo[1,5-c]pyrimidin-7-amine
   2H-1,2,4,6-thiatriazine-3,5-diamine 1,1-dioxide
   (2E)-2-(pyrid-2-ylmethylene)hydrazinecarboximidamide
   (2R)-2-{[amino(imino)methyl]amino}-3-sulfanylpropanoic acid
   triazane-2-carboximidohydrazide
   2-{[amino(imino)methyl]amino}ethanesulfonic acid
   2-amino-1,7-dihydro-6H-purine-6-thione
   N″-(4-chlorophenyl)guanidine
   N-[imino(morpholin-4-yl)methyl]guanidine
   6-{[amino(imino)methyl]amino}hexanoic acid
   (2S)-2-{[amino(imino)methyl]amino}succinic acid
   (2S)-5-{[amino(imino)methyl]amino}-2-hydroxypentanoic acid
   5-(propylsulfanyl)-1,2,4-thiadiazol-3-amine
   (2S)-2-amino-4-({[amino(imino)methyl]amino}oxy)butanoic acid
   (2S)-2-amino-4-{[(hydroxyamino)(imino)methyl]amino}butanoic acid
   4-{[amino(imino)methyl]amino}benzoic acid
   6-ethoxy-3,9-dihydro-2H-purin-2-imine
   N-(3-nitrophenyl)guanidine
   (2S)-2-amino-3-{[amino(imino)methyl]amino}propanoic acid
   N-(2-amino-6-chloropyrimidin-4-yl)guanidine
   N-(1,3-dimethyl-1,3-dihydro-2H-imidazol-2-ylidene)-N-phenylamine
   (2S)-2-amino-6-{[amino(imino)methyl]amino}hexanoic acid
   2-{[amino(imino)methyl]amino}pentanedioic acid
   (2S)-2-{[amino(imino)methyl]amino}pentanedioic acid
   [[amino(imino)methyl](2,3-dihydroxypropyl)amino]acetic acid
   4-hydroxy-3,4-dihydroisoquinoline-2(1H)-carboximidamide
   N-(2-methylphenyl)imidodicarbonimidic diamide
   N-(tert-butyl)-N″-phenylguanidine
   N5-phenyl-1,2,4-thiadiazole-3,5-diamine
   (2S)-5-{[amino(imino)methyl]amino}-2-chloropentanoic acid
   N-butylimidodicarbonimidic diamide
   N-(2-methyl-5-nitrophenyl)guanidine
   2-[(6-amino-1H-purin-8-yl)amino]ethanol
   N-[amino(imino)methyl]-N′-phenylthiourea
   2-hydrazino-1,4,5,6-tetrahydropyrimidine
   N′-nitro-2-phenylhydrazinecarboximidamide
   N-(4,6-dimethylpyrimidin-2-yl)-N″-methoxyguanidine
   6-(ethylsulfanyl)-1H-purin-2-amine
   (2S)-2-amino-4-{[amino(imino)methyl]amino}butanoic acid
   (2E)-2-(4-chlorobenzylidene)hydrazinecarboximidamide
   2-[amino(imino)methyl]-N-ethylhydrazinecarbothioamide
   4-({[amino(imino)methyl]amino}methyl)cyclohexanecarboxylic acid
   N-(1-naphthylmethyl)guanidine
   N,N″-dineopentylguanidine
   3,5-dimethyl-1H-pyrazole-1-carboximidamide
   N-(4-methylquinazolin-2-yl)guanidine
   N-(4-hydroxyquinazolin-2-yl)guanidine
   N-(1,3-dimethyltetrahydropyrimidin-2(1H)-ylidene)-N-phenylamine
   N-(4-chlorophenyl)guanidine
   (2E)-2-(1,3-benzodioxol-5-ylmethylene)hydrazinecarboximidamide
   N-(4,6-dimethylpyrimidin-2-yl)-N″-ethoxyguanidine
   N,N′-diphenylguanidine
   N-phenylimidodicarbonimidic diamide
   4-amino-N-[amino(imino)methyl]benzenesulfonamide
   ethyl 2-{[amino(imino)methyl]amino}-1,3-thiazole-4-carboxylate
   N-(4,6-dimethylquinazolin-2-yl)guanidine
   N-(4,7-dimethylquinazolin-2-yl)guanidine
   N-(4,8-dimethylquinazolin-2-yl)guanidine
   6,6-dimethyl-N2-phenyl-1,6-dihydro-1,3,5-triazine-2,4-diamine
   N-(tert-butyl)-N-(1,3-dimethyl-1,3-dihydro-2H-benzimidazol-2-ylidene)amine
   N-(3,4-dichlorobenzyl)guanidine
   N″-(4-phenyl-1,3-thiazol-2-yl)guanidine
   (3-ethyl-2-imino-2,3-dihydro-1H-benzimidazol-1-yl)acetic acid
   1H-perimidin-2-amine
   N-(tert-butyl)-N″-methoxy-N′-phenylguanidine
   5-(benzylsulfanyl)-1,2,4-thiadiazol-3-amine
   N-[2-(dimethylamino)-5-nitropyrimidin-4-yl]guanidine
   (1Z,2R)-2-amino-5-{[amino(imino)methyl]amino}-N-hydroxypentanimidic acid
   N″-(4-chlorophenyl)-N,N′-diethylguanidine
   (2E)-2-(pyrid-4-ylmethylene)hydrazinecarboximidamide
   N-[2-(3-chlorophenoxy)ethanimidoyl]guanidine
   N-(heptafluoropropyl)guanidine
   [(carboxymethyl)(methyl)amino](imino)methylamidophosphate
   1,3-dimethyl-2-(methylamino)-4,5-dihydro-1H-imidazole
   N-(4-methylphenyl)imidodicarbonimidic diamide
   N′-[(2Z)-1-methylpyrimidin-2(1H)-ylidene]benzohydrazide
   4-(aminomethyl)piperidine-1-carboximidamide
   N-(4,6-dimethylquinazolin-2-yl)-N″-methylguanidine
   N-(4,7-dimethylquinazolin-2-yl)-N″-methylguanidine
   5-{[amino(imino)methyl]amino}-2-chloropentanoic acid
   N-(2,6-dichlorophenyl)-4,5-dihydro-1H-imidazol-2-amine
   (2E)-2-(2,6-dichlorobenzylidene)hydrazinecarboximidamide
   (2E)-2-(2,4-dichlorobenzylidene)hydrazinecarboximidamide
   N-(6-methoxy-4-methylquinazolin-2-yl)guanidine
   N-(7-methoxy-4-methylquinazolin-2-yl)guanidine
   N-(8-methoxy-4-methylquinazolin-2-yl)guanidine
   6,6-dimethyl-N2-(4-methylphenyl)-1,6-dihydro-1,3,5-triazine-2,4-diamine
   N-(4-fluorophenyl)imidodicarbonimidic diamide
   N-(3-fluorophenyl)imidodicarbonimidic diamide
   N-(2-fluorophenyl)imidodicarbonimidic diamide
   N″-(3,4-dichlorophenyl)-N,N′-dimethylguanidine
   N-[2-(5-methoxy-1H-indol-3-yl)ethyl]guanidine
   N′-(1,3-dimethylimidazolidin-2-ylidene)benzohydrazide
   N-(5-bromo-4-methyl-1,3-thiazol-2-yl)guanidine
   2-[(2-{[amino(imino)methyl]amino}ethyl)sulfanyl]succinic acid
   N-(4,6-dimethylpyrimidin-2-yl)-N″-isopentylguanidine
   N-[(5-nitro-2-furyl)methyl]hydrazinecarboximidamide
   2,6-dimethylpiperidine-1-carboximidamide
   N-(1,3-dimethyl-1,3-dihydro-2H-benzimidazol-2-ylidene)-N-phenylamine
   3-[(2-{[amino(imino)methyl]amino}ethyl)sulfanyl]-2-(sulfanylmethyl)propanoic acid
   (E)-N′,2-diphenyldiazenecarbohydrazonamide
   N-(1,3-dimethyl-1,3-dihydro-2H-benzimidazol-2-ylidene)methanesulfonamide
   N,N′-bis(2-methylphenyl)guanidine
   sodium 4-[(2-{[amino(imino)methyl]amino}ethyl)sulfanyl]-4-oxobutanoate
   1,3-dimethyl-2-(methylamino)-3,4,5,6-tetrahydropyrimidine
   4-chloro-N-4,5-dihydro-1H-imidazol-2-yl-6-methoxy-2-methylpyrimidin-5-amine
   N-(2-phenylethyl)imidodicarbonimidic diamide
   N-(2,3-dimethylphenyl)imidodicarbonimidic diamide
   N-(4-hydroxy-6-methylpyrimidin-2-yl)-N′-phenylguanidine
   N9-phenyl-6,8,10-triazaspiro[4.5]deca-6,8-diene-7,9-diamine
   5-[(3aS,4S,6aR)-2-iminohexahydro-1H-thieno[3,4-d]imidazol-4-yl]pentanoic acid
   N-(3-methoxyphenyl)imidodicarbonimidic diamide
   N-(2-methoxyphenyl)imidodicarbonimidic diamide
   N-(4-methoxyphenyl)imidodicarbonimidic diamide
   (2E)-2-[1-(2,4-dichlorophenyl)ethylidene]hydrazinecarboximidamide
   N-(8-ethoxy-4-methylquinazolin-2-yl)guanidine
   N-(1,2-di-tert-butyidiaziridin-3-ylidene)-N-phenyl amine
   (2S)-3-{[amino(imino)methyl]amino}-2-[(tert-butoxycarbonyl)amino]propanoic acid
   N2-(2-methoxyphenyl)-6,6-dimethyl-1,6-dihydro-1,3,5-triazine-2,4-diamine
   N,N′-di-tert-butyl-N″-phenylguanidine
   N-(2-chlorophenyl)imidodicarbonimidic diamide
   N-(3-chlorophenyl)imidodicarbonimidic diamide
   N-(4-chlorophenyl)imidodicarbonimidic diamide
   N-1-naphthylguanidine
   N″-[4-(4-methoxyphenyl)-1,3-thiazol-2-yl]guanidine
   N-{imino[2-(1-methylethylidene)hydrazino]methyl}-N′-phenylthiourea
   N-(2,4-difluorophenyl)imidodicarbonimidic diamide
   N-(2,6-difluorophenyl)imidodicarbonimidic diamide
   N-(3,5-difluorophenyl)imidodicarbonimidic diamide
   N-(2,5-difluorophenyl)imidodicarbonimidic diamide
   N-(3,4-difluorophenyl)imidodicarbonimidic diamide
   (2E)-2-[4-(dimethylamino)-3-nitrobenzylidene]hydrazinecarboximidamide
   N2-(2-chlorophenyl)-6,6-dimethyl-1,6-dihydro-1,3,5-triazine-2,4-diamine
   N2-(4-chlorophenyl)-6,6-dimethyl-1,6-dihydro-1,3,5-triazine-2,4-diamine
   N-[4-(4-chlorophenyl)-1,3-thiazol-2-yl]guanidine
   N″-[4-(4-chlorophenyl)-1,3-thiazol-2-yl]guanidine
   6,6-dimethyl-1-phenyl-1,6-dihydro-1,3,5-triazine-2,4-diamine
   (1E)-1-(2-methylphenyl)ethanone[(2Z)-4,6-dimethylpyrimidin-2(1H)-ylidene]hydrazone
   (1Z)-1-(2-methylphenyl)ethanone[(2E)-4,6-dimethylpyrimidin-2(1H)-ylidene]hydrazone
   disodium[(carboxymethyl)(methyl)amino](imino)methyl amidophosphate
   N-(6-aminohexyl)guanidine
   (2 E)-2-((2E)-2-{[amino(imino)methyl]hydrazono}-1-methylethylidene)hydrazinecarboximidamide
   4-nitrobenzaldehyde[(2Z)-1-methylpyrimidin-2(1H)-ylidene]hydrazone
   4-{[{[amino(imino)methyl]amino}(imino)methyl]amino}benzenesulfonic acid
   N-1,3-benzodioxol-5-ylimidodicarbonimidic diamide
   N,3-di-tert-butyl-3,4-dihydroquinazolin-2-amine
   N-(4-fluorophenyl)-N′-(4-hydroxy-6-methylpyrimidin-2-yl)guanidine
   N-(2-chloro-6-methylphenyl)imidodicarbonimidic diamide
   N-hydroxyhydrazinecarboximidamide
   (2S)-2-amino-6-{[imino(methylamino)methyl]amino}hexanoic acid 1H-perimidin-2-amine
   N-[3-(1H-imidazol-4-yl)propyl]guanidine
   N-(tert-butyl)-5H-dibenzo[d,f][1,3]diazepin-6-amine
   N-(3-chloro-4-fluorophenyl)imidodicarbonimidic diamide
   N-(3-chloro-2-fluorophenyl)imidodicarbonimidic diamide
   (2-{[amino(imino)methyl]amino}-1,3-thiazol-4-yl)methylimidothiocarbamate
   N-(tert-butyl)-N′,N″-diphenylguanidine
   N-(1,2-dineopentyidiaziridin-3-ylidene)-N-neopentylamine
   N,N′,N″-trineopentylguanidine
   1-[bis(isopropylamino)methylene]-2-phenylhydrazine
   N″-(benzyloxy)-N-(4,6-dimethylpyrimidin-2-yl)guanidine
   N-(2-methyl-5-nitrophenyl)imidodicarbonimidic diamide
   methyl 2-amino-6-{[amino(imino)methyl]amino}hexanoate
   N-(4-hydroxy-6-methylpyrimidin-2-yl)-N′-(4-methoxyphenyl)guanidine
   N-[4-hydroxy-6-(methoxymethyl)pyrimidin-2-yl]-N′-phenylguanidine
   N-(4-hydroxy-6-methylpyrimidin-2-yl)-N′-(3-methoxyphenyl)guanidine
   N-(4-hydroxy-6-methylpyrimidin-2-yl)-N′-(2-methoxyphenyl)guanidine
   N,6-di-tert-butyl-8-methyl-3,4-dihydroquinazolin-2-amine
   N-(3,5-dimethoxyphenyl)imidodicarbonimidic diamide
   N-(3,4-dimethoxyphenyl)imidodicarbonimidic diamide
   N-[2-(5-methoxy-1H-indol-3-yl)ethyl]imidodicarbonimidic diamide
   4-{[amino(imino)methyl]amino}benzoic acid
   N-(4-chlorophenyl)-N′-(4,6-dimethylpyrimidin-2-yl)guanidine
   N,N′-di-tert-butyl-N″-[2-(hydroxymethyl)phenyl]guanidine
   N-(4-chlorophenyl)-N′-(4-hydroxy-6-methylpyrimidin-2-yl)guanidine
   N-(3-chlorophenyl)-N′-(4-hydroxy-6-methylpyrimidin-2-yl)guanidine
   N-(2-chlorophenyl)-N′-(4-hydroxy-6-methylpyrimidin-2-yl)guanidine
   N-(2,4-difluorophenyl)-N′-(4-hydroxy-6-methylpyrimidin-2-yl)guanidine
   N-(2,6-difluorophenyl)-N′-(4-hydroxy-6-methylpyrimidin-2-yl)guanidine
   (3-benzyl-2-imino-2,3-dihydro-1H-benzimidazol-1-yl)acetic acid
   N,N′-di-tert-butyl-N″-methylguanidine
   N,N″-di-tert-butyl-N′-methylguanidine
   N-[2-(trifluoromethyl)phenyl]imidodicarbonimidic diamide
   N-[3-(trifluoromethyl)phenyl]imidodicarbonimidic diamide
   N-[4-(trifluoromethyl)phenyl]imidodicarbonimidic diamide
   N-(2,3-dichlorophenyl)imidodicarbonimidic diamide
   N-(2,5-dichlorophenyl)imidodicarbonimidic diamide
   N-(3,4-dichlorophenyl)imidodicarbonimidic diamide
   N-(3,5-dichlorophenyl)imidodicarbonimidic diamide
   N-(2,4-dichlorophenyl)imidodicarbonimidic diamide
   4-{[[(4,6-dimethylpyrimidin-2-yl)amino](imino)methyl]amino}benzoic acid
   9,13b-dihydro-1H-dibenzo[c,f]imidazo[1,5-a]azepin-3-amine
   dipotassium[(carboxymethyl)(methyl)amino](imino)methylamidophosphate
   N,N′,N″-triphenylguanidine
   N-dodecylguanidine-N-(1,2-di-tert-butyldiaziridin-3-ylidene)-N-mesitylamine
   N,3-di-tert-butyl-6,8-dimethyl-3,4-dihydroquinazolin-2-amine
   N-(4-fluorophenyl)-N′-[3-hydroxy-5-(methoxymethyl)phenyl]guanidine
   N,N′-di-tert-butyl-N″-mesitylguanidine
   2-anilino-1,3-dimethyl-4,5-dihydro-1H-imidazole
   N-(4-fluorophenyl)-N′-[4-hydroxy-6-(methoxymethyl)pyrimidin-2-yl]guanidine
   N-(4-chlorophenyl)-N′-(4-hydroxy-5,6-dimethylpyrimidin-2-yl)guanidine
   5-bromo-N-4,5-dihydro-1H-imidazol-2-ylquinoxalin-6-amine
   N-(4-bromophenyl)imidodicarbonimidic diamide
   N,N′-dicyclohexylmorpholine-4-carboximidamide
   5-[(4S)-2-iminohexahydro-1H-thieno[3,4-d]imidazol-4-yl]pentanohydrazide
   N-(3-chloro-4-fluorophenyl)-N′-(4-hydroxy-6-methylpyrimidin-2-yl)guanidine
   N2-(4-bromophenyl)-6,6-dimethyl-1,6-dihydro-1,3,5-triazine-2,4-diamine
   (2S)-2-amino-4-{[(hydroxyamino)(imino)methyl]amino}butanoic acid
   N-(2-azocan-1-ylethyl)guanidine
   {[(E)-[(4-methylbenzyl)amino](phenylimino)methyl]amino}acetic acid
   N-[4-(trifluoromethoxy)phenyl]imidodicarbonimidic diamide
   4-nitrophenyl 4-[[amino(imino)methyl]amino}benzoate
   6-[(4-nitrobenzyl)sulfanyl]-1H-purin-2-amine
   N,N′,2-triphenylhydrazinecarboximidamide
   N-[4-hydroxy-6-(methoxymethyl)pyrimidin-2-yl]-N′-(3-methoxyphenyl)guanidine
   N-[4-hydroxy-6-(methoxymethyl)pyrimidin-2-yl]-N′-(4-methoxyphenyl)guanidine
   N-(3,4,5-trimethoxyphenyl)imidodicarbonimidic diamide
   N-[(1E)-hydrazino(2-phenylhydrazino)methylene]benzenesulfonamide
   N-[bis(neopentylamino)methylene]-2,2-dimethylpropan-1-amine
   5-[3-({imino[(2,2,2-trifluoroethyl)amino]methyl}amino)-1H-pyrazole-1-yl]pentanamide
   N-{3-[3-chloro-5-(trifluoromethyl)pyrid-2-yl]-1,2,4-oxadiazol-5-yl}guanidine
   N-(4-chlorophenyl)-N′-[4-hydroxy-6-(methoxymethyl)pyrimidin-2-yl]guanidine
   N-(2-chlorophenyl)-N′-[4-hydroxy-6-(methoxymethyl)pyrimidin-2-yl]guanidine
   N-(2,4-difluorophenyl)-N′-[4-hydroxy-6-(methoxymethyl)pyrimidin-2-yl]guanidine
   N,N′-di-tert-butyl-N″-isopropylguanidine
   5,8-di-tert-butyl-N-methyl-1H-perimidin-2-amine
   N-(4-hydroxy-6-methylpyrimidin-2-yl)-N′-[3-(trifluoromethyl)phenyl]guanidine
   N-(4-hydroxy-6-methylpyrimidin-2-yl)-N′-[4-(trifluoromethyl)phenyl]guanidine
   N-(3,4-dichlorophenyl)-N′-(4-hydroxy-6-methylpyrimidin-2-yl)guanidine
   N-(3,5-dichlorophenyl)-N′-(4-hydroxy-6-methylpyrimidin-2-yl)guanidine
   N-(2,3-dichlorophenyl)-N′-(4-hydroxy-6-methylpyrimidin-2-yl)guanidine
   1-[bis(isopropylamino)methylene]-1-isopropyl-2-phenylhydrazine
   N-{(5Z)-4-oxo-5-[2-(1H-1,2,4-triazol-1-yl)benzylidene]-4,5-dihydro-1,3-thiazol-2-yl}guanidine
   N-(4-hydroxy-5,6,7,8-tetrahydroquinazolin-2-yl)-N′-(3-methoxyphenyl)guanidine
   N-[(2Z)-1-methyl-4-phenylquinazolin-2(1H)-ylidene]methanesulfonamide
   N-(1,3-dimethyl-1,3-dihydro-2H-benzimidazol-2-ylidene)-4-methylbenzenesulfonamide
   N-(4-chlorophenyl)-N′-(4-hydroxy-5,6,7,8-tetrahydroquinazolin-2-yl)guanidine
   N-(2,4-difluorophenyl)-N′-(4-hydroxy-5,6,7,8-tetrahydroquinazolin-2-yl)guanidine
   N-(4-tert-butyl-6-hydroxypyrimidin-2-yl)-N′-(4-chlorophenyl)guanidine
   N-(4-tert-butyl-6-hydroxypyrimidin-2-yl)-N′-(2,4-difluorophenyl)guanidine
   ethyl 4-[(6-{[amino(imino)methyl]amino}hexanoyl)oxy]benzoate
   N-(tert-butyl)-N′,N″-bis(2,6-dimethylphenyl)guanidine
   N,N-bis(2-chlorobenzyl)-N″-hydroxyguanidine
   (2S)-6-{[amino(imino)methyl]amino}-2-[(tert-butoxycarbonyl)amino]hexanoic acid
   N-(3-chloro-4-fluorophenyl)-N′-[4-hydroxy-6-(methoxymethyl)pyrimidin-2-yl]guanidine
   N-(3-bromobenzyl)guanidine
   N-(4-hydroxy-6-methylpyrimidin-2-yl)-N′-[4-(trifluoromethoxy)phenyl]guanidine
   N-(4-tert-butyl-2,6-dimethylphenyl)-N-(1,2-di-tert-butyldiaziridin-3-ylidene)amine
   N-(2-{[amino(imino)methyl]amino}ethyl)isoquinoline-5-sulfonamide
   N-[4-hydroxy-6-(methoxymethyl)pyrimidin-2-yl]-N′-[3-(trifluoromethyl)phenyl]guanidine
   N-(3,4-dichlorophenyl)-N′-[4-hydroxy-6-(methoxymethyl)pyrimidin-2-yl]guanidine
   N-(3,5-dichlorophenyl)-N′-[4-hydroxy-6-(methoxymethyl)pyrimidin-2-yl]guanidine
   N-(tert-butyl)-N′-mesityl-N″-methylguanidine
   N,N″-di-tert-butyl-N′-phenylguanidine
   (2S)-2-[(tert-butoxycarbonyl)amino]-6-{[(diethylamino)-(imino)methyl]amino}hexanoic acid
   (2R)-2-[(tert-butoxycarbonyl)amino]-6-{[(diethylamino)-(imino)methyl]amino}hexanoic acid
   N-[3,5-bis(trifluoromethyl)phenyl]imidodicarbonimidic diamide
   3-(acetylamino)-4-{[amino(imino)methyl]amino}-2-(1,2,3-trihydroxypropyl)-3,4-dihydro-2H-pyran-6-carboxylic acid
   piperazine-1-carboximidamide
   N-{8-[(8-{[amino(imino)methyl]amino}octyl)amino]octyl}guanidine
   N-[4-hydroxy-6-(methoxymethyl)pyrimidin-2-yl]-N′-[4-(trifluoromethoxy)phenyl]guanidine
   N-{2-[(2-{[amino(imino)methyl]amino}ethyl)disulfanyl]ethyl}guanidine
   N-(4-methylphenyl)guanidine
   N-[amino(imino)methyl]-4-{[(1E)-3-(4-fluorophenyl)-3-oxoprop-1-enyl]amino}benzenesulfonamide
   4-benzyl-N-(3,5-dichlorophenyl)piperazine-1-carboximidamide
   N″-(tert-butyl)-N,N′-diphenylguanidine
   N-(tert-butyl)-N′-(2,6-diisopropyl-4-phenoxyphenyl)guanidine
   N,N″-di-tert-butyl-N′-(2,6-dimethylphenyl)guanidine
   N,N′,N″-tris(2,6-dimethylphenyl)guanidine
   (2S)-5-{[amino(imino)methyl]amino}-2-{[(benzoylamino)acetyl]oxy}pentanoic acid
   4-methyl-2-oxo-2H-chromen-7-yl 4-{[amino(imino)methyl]amino}benzoate
   N-(2,6-dichlorophenyl)-N-(4-fluorobenzyl)-4,5-dihydro-1H-imidazol-2-amine
   4-methylpiperazine-1-carboximidamide
   N-[2-(benzhydryloxy)ethyl]guanidine
   N-benzylguanidine
   N,N″-di-tert-butyl-N′-(4-tert-butylphenyl)guanidine
   N-(2-chlorophenyl)guanidine
   N-(2-chloroethyl)-N-[3,5-dichloro-4-(imidazolidin-2-ylideneamino)benzyl]-N-methylamine
   barium 2-(5-{[amino(imino)methyl]amino}-2-hydroxypentanoyl)succinate
   N,N″-di-tert-butyl-N′-(4-tert-butyl-2-methylphenyl)guanidine
   tert-butyl(1S)-1-(hydroxymethyl)-4-[(imino{[(4-methyl-phenyl)sulfonyl]amino}methyl)amino]butylcarbamate
   N-{8-[(8-{[amino(imino)methyl]amino}octyl)amino]octyl}guanidine
   ethyl 4-[(6-{[amino(imino)methyl]amino}hexanoyl)oxy]benzoate
   N″-(tert-butyl)-N,N′-bis(2,6-dimethylphenyl)guanidine
   7-{[amino(imino)methyl]amino}-N-[2-({4-[(3-aminopropyl)amino]butyl}amino)-1-hydroxy-2-oxoethyl]heptanamide
   N,N″-di-tert-butyl-N′-(4-tert-butyl-2,6-dimethylphenyl)guanidine
   N-octylguanidine
   3,4-dihydroisoquinoline-2(1H)-carboximidamide
   N-(2,5-dimethoxyphenyl)guanidine
   [[imino(phosphonoamino)methyl](methyl)amino]acetic acid
   N-(2-{[(E)-(tert-butylamino)(tert-butylimino)methyl]amino}benzyl)-N,N-diethylethanamine
   N-[3-(trifluoromethyl)phenyl]guanidine
   N-{bis[(2,6-dimethylphenyl)amino]methylene}-2,6-dimethylbenzeneamine
   4-{2-[2-(dimethylamino)-2-oxoethoxy]-2-oxoethyl}phenyl 4-{[amino(imino)methyl]amino}benzoate
   N-(4-chlorophenyl)-N′-(6-{[{[[(4-chlorophenyl)amino]-(imino)methyl]amino}(imino)methyl]amino}hexyl)imidodicarbonimidic diamide
   N-(2,3-dihydro-1,4-benzodioxin-2-ylmethyl)guanidine
   N-(2-{[(E)-(tert-butylamino)(tert-butylimino)methyl]amino}benzyl)-N,N-diethylethanamine
   6-[amino(imino)methyl]-2-naphthyl 4-{[amino(imino)methyl]amino}benzoate dimethanesulfonate
   N,N′dicyclohexylguanidine
   N-(4-chlorophenyl)-N′-(6-{[{[[(4-chlorophenyl)amino](imino)methyl]amino}(imino)methyl]amino}hexyl)imidodicarbonimidic diamide
   N-(4-chlorophenyl)-N′-(6-{[{[[(4-chlorophenyl)amino]-(imino)methyl]amino}(imino)methyl]amino}hexyl)imidodicarbonimidic diamide
   N-(2-ethylhexyl)-N′-(6-{[{[[(2-ethylhexyl)amino](imino)methyl]amino}(imino)methyl]amino}hexyl)imidodicarbonimidic diamide
   n-propylguanidine
   isopropylguanidine
   n-butylguanidine
   (2-methylpropyl)guanidine
   t-butylguanidine
   n-pentylguanidine
   n-hexylguanidine
   n-octylguanidine
   (2-ethylhexyl)guanidine
   n-decylguanidine
   n-dodecylguanidine
   (2-hydroxyethyl)guanidine
   (3-hydroxypropyl)guanidine
   (2-hydroxypropyl)guanidine
   (4-hydroxybutyl)guanidine
   (5-hydroxypentyl)guanidine
   (6-hydroxyhexyl)guanidine
   (1-hydroxymethylpentyl)guanidine
   (2-methoxyethyl)guanidine
   (2-ethoxyethyl)guanidine
   (3-methoxypropyl)guanidine
   (3-ethoxypropyl)guanidine
   (3-propyloxypropyl)guanidine
   (3-butyloxypropyl)guanidine
   (3-pentyloxypropyl)guanidine
   (3-hexyloxypropyl)guanidine
   guanidinoacetic acid
   3-guanidinopropanoic acid
   4-guanidinobutanoic acid
   5-guanidinopentanoic acid
   6-guanidinohexanoic acid
   methyl guanidinoacetate
   ethyl guanidinoacetate
   propyl guanidinoacetate
   methyl 3-guanidinopropanoate
   ethyl 3-guanidinopropanoate
   isopropyl 3-guanidinopropanoate
   methyl 4-guanidinobutanoate
   ethyl 4-guanidinobutanoate
   methyl 5-guanidinopentanoate
   ethyl 5-guanidinopentanoate
   methyl 6-guanidinohexanoate
   ethyl 6-guanidinohexanoate
   isopropyl 6-guanidinohexanoate
   o-, m- or p-hydroxyphenylguanidine
   o- or m-methylphenylguanidine
   o-, m- or p-methoxyphenylguanidine
   o-, m- or p-ethoxyphenylguanidine
   o-, m- or p-guanidinobenzoic acid
   methyl o-, m- or p-guanidinobenzoate
   ethyl o-, m- or p-guanidinobenzoate
   o-, m- or p-guanidinophenyl acetate
   o-, m- or p-guanidinophenyl propanoate
   o-, m- or p-guanidinophenylacetic acid
   o-, m- or p-guanidinophenylpropanoic acid
   o-, m- or p-guanidinophenylbutanoic acid
   o-, m- or p-guanidinophenylpentanoic acid
   o-, m- or p-guanidinophenylhexanoic acid
   phenethylguanidine
   naphthylguanidine
   o-, m- or p-hydroxybenzylguanidine
   o-, m- or p-methylbenzylguanidine
   o-, m- or p-methoxybenzylguanidine
   o-, m- or p-ethoxybenzylguanidine
   o-, m- or p-guanidinomethylbenzoic acid
   methyl o-, m- or p-guanidinomethylbenzoate
   ethyl o-, m- or p-guanidinomethylbenzoate
   isopropyl o-, m- or p-guanidinomethylbenzoate
   cyclopentylguanidine
   cyclohexylguanidine
   cyclohexylmethylguanidine
   4-guanidinomethylcyclohexanecarboxylic acid
   2-guanidinocyclohexanecarboxylic acid
   3-guanidinocyclohexanecarboxylic acid
   4-guanidinocyclohexanecarboxylic acid
   methyl 2-guanidinocyclohexanecarboxylate
   ethyl 2-guanidinocyclohexanecarboxylate
   4-oxacyclohexylguanidine
   glucopyranosylguanidine
   deoxyguanidinoinositol
   2-(2-hydroxyethoxy)ethylguanidine
   2-[2-(2-hydroxy)ethoxy]ethylguanidine
   2-(2-methoxyethoxy)ethylguanidine
   2-(2-ethoxyethoxy)ethylguanidine
   2-[2-(2-methoxyethoxy)ethyl]guanidine
   2-[2-(2-ethoxyethoxy)ethoxy]ethylguanidine
   2-guanidinoethanesulfonic acid
   3-guanidinopropanesulfonic acid
   4-guanidinobutanesulfonic acid
   5-guanidinopentanesulfonic acid
   6-guanidinohexanesulfonic acid
   3-guanidino-2-hydroxy-1-propanesulfonic acid
   2-guanidinoethyl dihydrogenophosphate
   3-guanidinopropyl dihydrogenophosphate
   4-guanidinobutyl dihydrogenophosphate
   5-guanidinopentyl dihydrogenophosphate
   6-guanidinohexyl dihydrogenophosphate
   biguanido(guanylguanidine)
   1-methylbiguanide
   1-ethylbiguanide
   1-n-propylbiguanide
   1-isopropylbiguanide
   1-n-butylbiguanide
   1-(2-methylpropyl)biguanide
   1-t-butylbiguanide
   1-n-pentylbiguanide
   1-n-hexylbiguanide
   1-n-octylbiguanide
   1-(2-ethylhexyl)biguanide
   1-n-decylbiguanide
   1-n-dodecylbiguanide
   1-(2-hydroxyethyl)biguanide
   1-(3-hydroxypropyl)biguanide
   1-(2-hydroxypropyl)biguanide
   1-(4-hydroxybutyl)biguanide
   1-(5-hydroxypentyl)biguanide
   1-(6-hydroxyhexyl)biguanide
   1-(1-hydroxymethylpentyl)biguanide
   1-(2-methoxyethyl)biguanide
   1-(2-ethoxyethyl)biguanide
   1-(3-methoxypropyl)biguanide
   1-(3-ethoxypropyl)biguanide
   1-(3-propyloxypropyl)biguanide
   1-(3-butyloxypropyl)biguanide
   1-(3-pentyloxypropyl)biguanide
   1-(3-hexyloxypropyl)biguanide
   1-phenylbiguanide
   1-( o-, m- or p-hydroxyphenyl)biguanide
   1-( o-, m- or p-methylphenyl)biguanide
   o-, m- or p-methoxyphenylbiguanide
   o-, m- or p-ethoxyphenylbiguanide
   1-benzylbiguanide
   1-cyclopentylbiguanide
   1-cyclohexylbiguanide
   1-(4-oxacyclohexyl)biguanide
   1-[2-(2-hydroxyethoxy)ethyl]biguanide
   1-[2-[2-(2-hydroxyethoxy)ethoxy]ethyl]biguanide
   1,1-dimethylbiguanide
   1,1-diethylbiguanide
   1-ethyl-1-methylbiguanide
   1,1-dipropylbiguanide
   1,1-dibutylbiguanide
   1,1-di-n-propylguanidine
   1,1-diisopropylguanidine
   1,1-di-n-butylguanidine
   1,1-bis(2-methylpropyl)guanidine
   1,1-di-t-butylguanidine
   1,1-di-n-pentylguanidine
   1,1-bis(2-ethylhexyl)guanidine
   1,1-di-n-hexylguanidine
   1,1-dioctylguanidine
   1,1-di-n-decylguanidine
   1,1-di-n-dodecylguanidine
   1-methyl-1-ethylguanidine
   1-methyl-1-n-propylguanidine
   1-methyl-1-isopropylguanidine
   1-methyl-1-n-butylguanidine
   1-methyl-1-(2-methylpropyl)guanidine
   1-methyl-1-t-butylguanidine
   1-methyl-1-n-pentylguanidine
   1-methyl-1-(2-ethylhexyl)guanidine
   1-methyl-1-n-hexylguanidine
   1-methyl-1-n-octylguanidine
   1-methyl-1-n-decylguanidine
   1-methyl-1-n-dodecylguanidine
   1,1-bis(2-hydroxyethyl)guanidine
   1,1-bis(3-hydroxypropyl)guanidine
   1,1-bis(2-hydroxypropyl)guanidine
   1,1-bis(4-hydroxybutyl)guanidine
   1,1-bis(5-hydroxypentyl)guanidine
   1,1-bis(6-hydroxyhexyl)guanidine
   1,1-bis(1-hydroxymethylpentyl)guanidine
   1-methyl-1-(2-hydroxyethyl)guanidine
   1-methyl-1-(3-hydroxypropyl)guanidine
   1-methyl-1-(2-hydroxypropyl)guanidine
   1-methyl-1-(4-hydroxybutyl)guanidine
   1-methyl-1-(5-hydroxypentyl)guanidine
   1-methyl-1-(6-hydroxyhexyl)guanidine
   1-methyl-1-(1-hydroxymethylpentyl)guanidine
   1,1-bis(2-methoxyethyl)guanidine
   1,1-bis(2-ethoxyethyl)guanidine
   1,1-bis(3-methoxypropyl)guanidine
   1,1-bis(3-ethoxypropyl)guanidine
   1,1-bis(3-propyloxypropyl)guanidine
   1,1-bis(3-butyloxypropyl)guanidine
   1,1-bis(3-pentyloxypropyl)guanidine
   1,1-bis(3-hexyloxypropyl)guanidine
   1-methyl-1-(2-methoxyethyl)guanidine
   1-methyl-1-(2-ethoxyethyl)guanidine
   1-methyl-1-(3-methoxypropyl)guanidine
   1-methyl-1-(3-ethoxypropyl)guanidine
   1-methyl-1-(3-propyloxypropyl)guanidine
   1-methyl-1-(3-butyloxypropyl)guanidine
   1-methyl-1-(3-pentyloxypropyl)guanidine
   1-methyl-1-(3-hexyloxypropyl)guanidine
   1-methylguanidinoacetic acid
   3-(1-methylguanidino)propanoic acid
   4-(1-methylguanidino)butanoic acid
   5-(1-methylguanidino)pentanoic acid
   6-(1-methylguanidino)hexanoic acid
   methyl 1-methylguanidinoacetate
   ethyl 1-methylguanidinoacetate
   propyl 1-methylguanidinoacetate
   methyl 3-(1-methylguanidino)propanoate
   ethyl 3-(1-methylguanidino)propanoate
   propyl 3-(1-methylguanidino)propanoate
   isopropyl 3-(1-methylguanidino)propanoate
   methyl 4-(1-methylguanidino)butanoate
   methyl 5-(1-methylguanidino)pentanoate
   ethyl 5-(1-methylguanidino)pentanoate
   methyl 6-(1-methylguanidino)hexanoate
   ethyl 6-(1-methylguanidino)hexanoate
   1,1-diphenylguanidine
   1,1-bis(o-, m- or p-hydroxyphenyl)guanidine
   1,1-bis(o-, m- or p-methylphenyl)guanidine
   1,1-bis(o-, m- or p-methoxyphenyl)guanidine
   1,1-bis(o-, m- or p-ethoxyphenyl)guanidine
   1-methyl-1-phenylguanidine
   1-methyl-1-( o-, m- or p-hydroxyphenyl)guanidine
   1-methyl-1-( o-, m- or p-methylphenyl)guanidine
   1-methyl-1-( o-, m- or p-methoxyphenyl)guanidine
   1-methyl-1-( o-, m- or p-ethoxyphenyl)guanidine
   o-, m- or p-(1-methylguanidino)benzoic acid
   methyl o-, m- or p-(1-methylguanidino)benzoate
   ethyl o-, m- or p-(1-methylguanidino)benzoate
   o-, m- or p-(1-methylguanidino)phenyl acetate
   o-, m- or p-(1-methylguanidino)phenyl propanoate
   o-, m- or p-(1-methylguanidino)phenylacetic acid
   o-, m- or p-(1-methylguanidino)phenylpropanoic acid
   o-, m- or p-(1-methylguanidino)phenylbutanoic acid
   o-, m- or p-(1-methylguanidino)phenylpentanoic acid
   o-, m- or p-(1-methylguanidino)phenylhexanoic acid
   1,1-dibenzylguanidine
   1,1-bis[o-, m- or p-(hydroxybenzyl)]guanidine
   1,1-bis[o-, m- or p-(methylbenzyl)]guanidine
   1,1-bis[o-, m- or p-(methoxybenzyl)]guanidine
   1,1-bis[o-, m- or p-(ethoxybenzyl)]guanidine
   1-methyl-1-benzylguanidine
   1-methyl-1-[o-, m- or p-(hydroxybenzyl)]guanidine
   1-methyl-1-[o-, m- or p-(methylbenzyl)]guanidine
   1-methyl-1-[o-, m- or p-(methoxybenzyl)]guanidine
   1-methyl-1-[o-, m- or p-(ethoxybenzyl)]guanidine
   o-, m- or p-(1-methylguanidinomethyl)benzoic acid
   methyl o-, m- or p-(1-methylguanidinomethyl)benzoate
   ethyl o-, m- or p-(1-methylguanidinomethyl)benzoate
   isopropyl o-, m- or p-(1-methylguanidinomethyl)benzoate
   1,1-dicyclopentylguanidine
   1,1-dicyclohexylguanidine
   1-methyl-1-cyclopentylguanidine
   1-methyl-1-cyclohexylguanidine
   2-(1-methylguanidino)cyclohexanecarboxylic acid
   3-(1-methylguanidino)cyclohexanecarboxylic acid
   4-(1-methylguanidino)cyclohexanecarboxylic acid
   methyl 2-(1-methylguanidino)cyclohexanecarboxylate
   ethyl 2-(1-methylguanidino)cyclohexanecarboxylate
   1,1-bis(4-oxacyclohexyl)guanidine
   1,1-di(glucopyranosyl)guanidine
   1-methyl-1-(4-oxacyclohexyl)guanidine
   1-methyl-1-(glucopyranosyl)guanidine
   1,1-bis[2-(2-hydroxyethoxy)ethyl]guanidine
   1,1-bis[2-[2-(2-hydroxyethoxy)ethoxy]ethyl]guanidine
   1-methyl-1-[2-(2-hydroxyethoxy)ethyl]guanidine
   1-methyl-1-[2-(2-hydroxyethoxy)ethoxy]ethylguanidine
   1,1-bis[2-(2-methoxyethoxy)ethyl]guanidine
   1,1-bis[2-(2-ethoxyethoxy)ethyl]guanidine
   1,1-bis[2-[2-(2-methoxyethoxy)ethoxy]ethyl]guanidine
   1,1-bis[2-[2-(2-ethoxyethoxy)ethoxy]ethyl]guanidine
   1-methyl-1-[2-(2-methoxyethoxy)ethyl]guanidine
   1-methyl-1-[2-(2-ethoxyethoxy)ethyl]guanidine
   1-methyl-1-[2-[2-(2-methoxyethoxy)ethoxy]ethyl]guanidine
   1-methyl-1-[2-[2-(2-ethoxyethoxy)ethoxy]ethyl]guanidine
   1,3-dimethylguanidine
   1,3-di-n-propylguanidine
   1,3-di-n-butylguanidine
   1,3-bis(2-methylpropyl)guanidine
   1,3-di-t-butylguanidine
   1,3-di-n-pentylguanidine
   1,3-bis(2-ethylhexyl)guanidine
   1,3-di-n-hexylguanidine
   1-dioctylguanidine
   1,3-di-n-decylguanidine
   1,3-di-n-dodecylguanidine
   3-methyl-1-ethylguanidine
   3-methyl-1-n-propylguanidine
   3-methyl-1-isopropylguanidine
   3-methyl-1-n-butylguanidine
   3-methyl-1-(2-methylpropyl)guanidine
   3-methyl-1-t-butylguanidine
   3-methyl-1-n-pentylguanidine
   3-methyl-1-(2-ethylhexyl)guanidine
   3-methyl-1-n-hexylguanidine
   3-methyl-1-n-octylguanidine
   3-methyl-1-n-decylguanidine
   3-methyl-1-n-dodecylguanidine
   1,3-bis(2-hydroxyethyl)guanidine
   1,3-bis(3-hydroxypropyl)guanidine
   1,3-bis(2-hydroxypropyl)guanidine
   1,3-bis(4-hydroxybutyl)guanidine
   1,3-bis(5-hydroxypentyl)guanidine
   1,3-bis(6-hydroxyhexyl)guanidine
   1,3-bis(1-hydroxymethylpentyl)guanidine
   3-methyl-1-(2-hydroxyethyl)guanidine
   3-methyl-3-hydroxypropyl)guanidine
   3-methyl-1-(2-hydroxypropyl)guanidine
   3-methyl-1-(4-hydroxybutyl)guanidine
   3-methyl-1-(5-hydroxypentyl)guanidine
   3-methyl-1-(6-hydroxyhexyl)guanidine
   3-methyl-1-(hydroxymethylpentyl)guanidine
   1,3-bis(2-methoxyethyl)guanidine
   1,3-bis(2-ethoxyethyl)guanidine
   1,3-bis(3-methoxypropyl)guanidine
   1,3-bis(3-ethoxypropyl)guanidine
   1,3-bis(3-propyloxypropyl)guanidine
   1,3-bis(3-butyloxypropyl)guanidine
   1,3-bis(3-pentyloxypropyl)guanidine
   1,3-bis(3-hexyloxypropyl)guanidine
   3-methyl-1-(2-methoxyethyl)guanidine
   3-methyl-1-(2-ethoxyethyl)guanidine
   3-methyl-1-(3-methoxypropyl)guanidine
   3-methyl-1-(3-ethoxypropyl)guanidine
   3-methyl-1-(3-propyloxypropyl)guanidine
   3-methyl-1-(3-butyloxypropyl)guanidine
   3-methyl-1-(3-pentyloxypropyl)guanidine
   3-methyl-1-(3-hexyloxypropyl)guanidine
   3-methylguanidinoacetic acid
   3-(3-methylguanidino)propanoic acid
   4-(3-methylguanidino)butanoic acid
   5-(3-methylguanidino)pentanoic acid
   6-(3-methylguanidino)hexanoic acid
   methyl 3-methylguanidinoacetate
   ethyl 3-methylguanidinoacetate
   propyl 3-methylguanidinoacetate
   methyl 3-(3-methylguanidino)propanoate
   ethyl 3-(3-methylguanidino)propanoate
   propyl 3-(3-methylguanidino)propanoate
   isopropyl 3-(3-methylguanidino)propanoate
   methyl 4-(3-methylguanidino)butanoate
   ethyl 4-(3-methylguanidino)butanoate
   propyl 4-(3-methylguanidino)butanoate
   isopropyl 4-(3-methylguanidino)butanoate
   methyl 5-(3-methylguanidino)pentanoate
   ethyl 5-(3-methylguanidino)pentanoate
   methyl 6-(3-methylguanidino)hexanoate
   ethyl 6-(3-methylguanidino)hexanoate
   1,3-bis(o-, m- or p-hydroxyphenyl)guanidine
   1,3-bis(m- or p-methylphenyl)guanidine
   1,3-bis(o-, m- or p-methoxyphenyl)guanidine
   1,3-bis(o-, m- or p-ethoxyphenyl)guanidine
   3-methyl-1-phenylguanidine
   3-methyl-1-(o-, m- or p-hydroxyphenyl)guanidine
   3-methyl-1-(o-, m- or p-methylphenyl)guanidine
   3-methyl-1-(o-, m- or p-methoxyphenyl)guanidine
   3-methyl-1-(o-, m- or p-ethoxyphenyl)guanidine
   o-, m- or p-(3-methylguanidino)benzoic acid
   methyl o-, m- or p-(3-methylguanidino)benzoate
   ethyl o-, m- or p-(3-methylguanidino)benzoate
   o-, m- or p-(3-methylguanidino)phenyl acetate
   o-, m- or p-(3-methylguanidino)phenyl propanoate
   o-, m- or p-(3-methylguanidino)phenylacetic acid
   o-, m- or p-(3-methylguanidino)phenylpropanoic acid
   o-, m- or p-(3-methylguanidino)phenylbutanoic acid
   o-, m- or p-(3-methylguanidino)phenylpentanoic acid
   o-, m- or p-(3-methylguanidino)phenylhexanoic acid
   1,3-dibenzylguanidine
   1,3-bis[o-, m- or p-(hydroxybenzyl)]guanidine
   1,3-bis[o-, m- or p-(methylbenzyl)]guanidine
   1,3-bis[o-, m- or p-(methoxybenzyl)]guanidine
   1,3-bis[o-, m- or p-(ethoxybenzyl)]guanidine
   3-methyl-1-benzylguanidine
   3-methyl-1-o-, m- or p-(hydroxybenzyl)guanidine
   3-methyl-1-o-, m- or p-(methylbenzyl)guanidine
   3-methyl-1-o-, m- or p-(methoxybenzyl)guanidine
   3-methyl-1-o-, m- or p-(ethoxybenzyl)guanidine
   o-, m- or p-(3-methylguanidinomethyl)benzoic acid
   methyl o-, m- or p-(3-methylguanidinomethyl)benzoate
   ethyl o-, m- or p-(3-methylguanidinomethyl)benzoate
   isopropyl o-, m- or p-(3-methylguanidinomethyl)benzoate
   1,3-dicyclopentylguanidine
   1,3-dicyclohexylguanidine
   3-methyl-1-cyclopentylguanidine
   3-methyl-1-cyclohexylguanidine
   2-(3-methylguanidino)cyclohexanecarboxylic acid
   3-(3-methylguanidino)cyclohexanecarboxylic acid
   4-(3-methylguanidino)cyclohexanecarboxylic acid
   methyl 2-(3-methylguanidino)cyclohexanecarboxylate
   ethyl 2-(3-methylguanidino)cyclohexanecarboxylate
   1,3-bis(4-oxacyclohexyl)guanidine
   1,3-bis(glucopyranosyl)guanidine
   3-methyl-1-(4-oxacyclohexyl)guanidine
   3-methyl-1-(glucopyranosyl)guanidine
   1,3-bis[2-(2-hydroxyethoxy)ethyl]guanidine
   1,3-bis[2-[2-(2-hydroxyethoxy)ethoxy]ethyl]guanidine
   3-methyl-1-[2-(2-hydroxyethoxy)ethyl]guanidine
   3-methyl-1-[2-(2-hydroxyethoxy)ethoxy]ethylguanidine
   1,3-bis[2-(2-methoxyethoxy)ethyl]guanidine
   1,3-bis[2-(2-ethoxyethoxy)ethyl]guanidine
   1,3-bis[2-[2-(2-methoxyethoxy)ethoxy]ethyl]guanidine
   1,3-bis[2-[2-(2-methoxymethoxy)ethoxy]ethyl]guanidine
   3-methyl-1-[2-(2-methoxyethoxy)ethyl]guanidine
   3-methyl-1-[2-(2-ethoxyethoxy)ethyl]guanidine
   3-methyl-1-[2-[2-(2-methoxyethoxy)ethoxy]ethyl]guanidine
   3-methyl-1-[2-[2-(2-ethoxyethoxy)ethoxy]ethyl]guanidine, and
   N-[bis(dimethylamino)methylene]-N′,N″-diisopropylguanidine
(b) In some embodiments, the guanidine-type imines may be chosen from:
   N,N-dimethylguanidine
   N,N′,N″-trimethylguanidine
   N-methylguanidine
   N,N-diethylguanidine
   N-ethylguanidine
   N,N-dimethylimidodicarbonimidic diamide
   N-(tert-butyl)-N″-methylguanidine
   N-(4-aminobutyl)guanidine
   1,3,4,6,7,8-hexahydro-2H-pyrimidol[1,2-a]pyrimidine
   N,N″-diisopropylguanidine
   1-methyl-1,3,4,6,7,8-hexahydro-2H-pyrimidol[1,2a]pyrimidine
   N″-(tert-butyl)-N,N,N′,N′-tetramethylguanidine
   N,N,N′,N′-tetraethylguanidine
   N,N″-di-tert-butylguanidine
   N″-[amino(imino)methyl]-N-phenylguanidine
   N,N′,N″-triisopropylguanidine
   pyrrolidine-1-carboximidamide
   N-phenylguanidine
   N-(tert-butyl)-N′,N″-dimethylguanidine
   N-(4-{[amino(imino)methyl]amino}butyl)guanidine
   N″-(tert-butyl)-N-methyl-N′-phenylguanidine
   N,N′,N″-tricyclohexylguanidine
   N-{3-[(4{[amino(imino)methyl]amino}butyl)amino]propyl}guanidine
   N-(2-hydroxyethyl)-N-methylguanidine
   N-(10-{[amino(imino)methyl]amino}decyl)guanidine
   n-propylguanidine
   isopropylguanidine
   n-butylguanidine
   (2-methylpropyl)guanidine
   t-butylguanidine
   n-pentylguanidine
   n-hexylguanidine
   (2-ethylhexyl)guanidine
   (2-hydroxyethyl)guanidine
   (3-hydroxypropyl)guanidine
   (2-hydroxypropyl)guanidine
   (4-hydroxybutyl)guanidine
   (5-hydroxypentyl)guanidine
   (6-hydroxyhexyl)guanidine
   (1-hydroxymethylpentyl)guanidine
   (2-methoxyethyl)guanidine
   (2-ethoxyethyl)guanidine
   (3-methoxypropyl)guanidine
   (3-ethoxypropyl)guanidine
   o-, m- or p-hydroxyphenylguanidine
   o-, m-methylphenylguanidine
   o-, m- or p-methoxyphenylguanidine
   phenethylguanidine
   o-, m- or p-hydroxybenzylguanidine
   o-, m- or p-methylbenzylguanidine
   o-, m- or p-methoxybenzylguanidine
   cyclopentylguanidine
   cyclohexylguanidine
   cyclohexylmethylguanidine
   4-oxacyclohexylguanidine
   glucopyranosylguanidine
   deoxyguanidinoinositol
   2-(2-hydroxyethoxy)ethylguanidine
   2-[2-(2-hydroxy)ethoxy]ethylguanidine
   2-(2-methoxyethoxy)ethylguanidine
   2-(2-ethoxyethoxy)ethylguanidine
   2-[2-(2-methoxyethoxy)ethyl]guanidine
   2-[2-(2-ethoxyethoxy)ethoxy]ethylguanidine
   biguanido(guanylguanidine)
   1-methylbiguanide
   1-ethylbiguanide
   1-n-propylbiguanide
   1-isopropylbiguanide
   1-n-butylbiguanide
   1-(2-methylpropyl)biguanide
   1-t-butylbiguanide
   1-n-pentylbiguanide
   1-n-hexylbiguanide
   1-(2-hydroxyethyl)biguanide
   1-(3-hydroxypropyl)biguanide
   1-(2-hydroxypropyl)biguanide
   1-(4-hydroxybutyl)biguanide
   1-phenylbiguanide
   1-benzylbiguanide
   1-cyclopentylbiguanide
   1-cyclohexylbiguanide
   1-4-oxacyclohexylbiguanide
   1,1-dimethylbiguanide
   1,1-diethylbiguanide
   1-ethyl-1-methylbiguanide
   1,1-di-n-propylguanidine
   1,1-diisopropylguanidine
   1,1-di-n-butylguanidine
   1,1-bis(2-methylpropyl)guanidine
   1,1-di-t-butylguanidine
   1-methyl-1-ethylguanidine
   1-methyl-1-n-propylguanidine
   1-methyl-1-isopropylguanidine
   1-methyl-1-n-butylguanidine
   1,1-bis(2-hydroxyethyl)guanidine
   1,1-bis(3-hydroxypropyl)guanidine
   1,1-bis(2-hydroxypropyl)guanidine
   1-methyl-1-(2-hydroxyethyl)guanidine
   1-methyl-1-(3-hydroxypropyl)guanidine
   1-methyl-1-(2-hydroxypropyl)guanidine
   1,1-diphenylguanidine
   1-methyl-1-phenylguanidine
   1,1-dibenzylguanidine
   1-methyl-1-benzylguanidine
   1,3-dimethylguanidine
   1,3-di-n-propylguanidine
   1,3-di-n-butylguanidine
   1,3-bis(2-methylpropyl)guanidine
   1,3-di-t-butylguanidine
   1,3-di-n-pentylguanidine
   1,3-bis(2-ethylhexyl)guanidine
   1,3-di-n-hexylguanidine
   3-methyl-1-ethylguanidine
   3-methyl-1-n-propylguanidine
   3-methyl-1-isopropylguanidine
   3-methyl-1-n-butylguanidine
   3-methyl-1-(2-methylpropyl)guanidine
   3-methyl-1-t-butylguanidine
   3-methyl-1-n-pentylguanidine
   1,3-bis(2-hydroxyethyl)guanidine
   1,3-bis(3-hydroxypropyl)guanidine
   1,3-bis(2-hydroxypropyl)guanidine
   3-methyl-1-(2-hydroxyethyl)guanidine
   3-methyl-(3-hydroxypropyl)guanidine
   3-methyl-1-(2-hydroxypropyl)guanidine
   1,3-dibenzylguanidine
   3-methyl-1-benzylguanidine
   1,3-dicyclopentylguanidine
   1,3-dicyclohexylguanidine
   3-methyl-1-cyclopentylguanidine
   3-methyl-1-cyclohexylguanidine
   1,3-di-(4-oxacyclohexyl)guanidine
   1,3-di-(glucopyranosyl)guanidine
   3-methyl-1-(4-oxacyclohexyl)guanidine
   3-methyl-1-(glucopyranosyl)guanidine, and
   N-[bis(dimethylamino)methylene]-N′,N″-diisopropylguanidine
(c) As a further example, the guanidine-type imines may be chosen from:
   N,N-dimethylguanidine
   N,N′,N″-trimethylguanidine
   N-methylguanidine
   N,N-diethylguanidine
   N-ethylguanidine
   N,N-dimethylimidodicarbonimidic diamide
   N-(tert-butyl)-N″-methylguanidine
   N-(4-aminobutyl)guanidine
   1,3,4,6,7,8-hexahydro-2H-pyrimido[1,2-a]pyrimidine
   N,N″-diisopropylguanidine
   1-methyl-1,3,4,6,7,8-hexahydro-2H-pyrimido[1,2-a]pyrimidine
   N″-(tert-butyl)-N,N,N′,N′-tetramethylguanidine
   N,N,N′,N′-tetraethylguanidine
   N,N″-di-tert-butylguanidine
   N″-[amino(imino)methyl]-N-phenylguanidine
   N,N′,N″-triisopropylguanidine
   pyrrolidine-1-carboximidamide
   N-phenylguanidine
   N-(tert-butyl)-N′,N″-dimethylguanidine
   N-(4-{[amino(imino)methyl]amino}butyl)guanidine
   N″-(tert-butyl)-N-methyl-N′-phenylguanidine
   N,N′,N″-tricyclohexylguanidine
   N-{3-[(4-{[amino(imino)methyl]amino}butyl)amino]propyl}guanidine
   N-(2-hydroxyethyl)-N-methylguanidine
   n-propylguanidine
   isopropylguanidine
   (2-methylpropyl)guanidine
   (2-ethylhexyl)guanidine
   (2-hydroxyethyl)guanidine
   (2-methoxyethyl)guanidine
   o-, m- or p-hydroxybenzylguanidine
   cyclopentylguanidine
   cyclohexylguanidine
   cyclohexylmethylguanidine
   4-oxacyclohexylguanidine
   glucopyranosylguanidine
   biguanido(guanylguanidine)
   1-methylbiguanide
   1-ethylbiguanide
   1-n-propylbiguanide
   1-isopropylbiguanide
   1-(2-methylpropyl)biguanide
   1-(2-hydroxyethyl)biguanide
   1-phenylbiguanide
   1-benzylbiguanide
   1-cyclopentylbiguanide
   1-cyclohexylbiguanide
   1,1-dimethylbiguanide
   1,1-diethylbiguanide
   1-ethyl-1-methylbiguanide
   1-methyl-1-ethylguanidine
   1-methyl-1-n-propylguanidine
   1-methyl-1-isopropylguanidine
   1,1-bis(2-hydroxyethyl)guanidine
   1-methyl-1-phenylguanidine
   3-methyl-1-ethylguanidine
   3-methyl-1-n-propylguanidine
   3-methyl-1-isopropylguanidine
   1,3-bis(2-hydroxyethyl)guanidine
   1,3-dibenzylguanidine
   3-methyl-1-benzylguanidine
   1,3-dicyclopentylguanidine
   3-methyl-1-cyclopentylguanidine
   3-methyl-1-cyclohexylguanidine
   1,3-di-(4-oxacyclohexyl)guanidine
   3-methyl-1-(4-oxacyclohexyl)guanidine
   3-methyl-1-(glucopyranosyl)guanidine, and
   N-[bis(dimethylamino)methylene]-N′,N″-diisopropylguanidine.

(2) Amidine Type Imines,
(a) such as:
   (3,3-dimethyl-3H-indol-2-yl)methylamine
   1,2,3,4,4a,5,6,7-octahydro-2,2,4a,7,7-pentamethyinaphthyridine
   1,2,3,9-tetrahydropyrrolo[2,1-b]quinazolin-9-ol
   1,2,3,9-tetrahydropyrrolo[2,1-b]quinazoline
   1,3-benzodioxole-5-carboximidamide
   1,3-benzodioxole-5-ethanimidamide
   1,4-benzodioxan-1-carboxamidine
   1-benzothiophene-3-carboximidamide
   1-benzyl-2,4-dioxohexahydro-5-pyrimidinecarboximidamide
   1-methyl-2-(2-thiophen-2-ylvinyl)-1,4,5,6-tetrahydro-pyrimidine
   1-methyl-2,4-dioxohexahydro-5-pyrimidinecarboximidamide
   1-methyl-2-imino-1,2-dihydropyridine
   1-methylquinolin-2(1H)-imine
   1-piperidinepropanimidamide
   2-(1H-1,2,4-triazol-1-yl)ethanimidamide
   2-(2,2-dimethylpropyl)-4,5-dihydro-1H-imidazole
   2-(2,3-dihydrobenzo[1,4]dioxin-2-yl)-4,5-dihydro-1H-imidazole
   2-(2,3-dihydrobenzo[1,4]dioxin-2-yl)-4,5-dihydro-1H-imidazole
   2-(2-benzofuranyl)-2-imidazoline
   2-(2-chlorophenoxy)ethanimidamide
   2-(4,5-dihydro-1H-imidazol-2-yl)-6-methoxypyrazine
   2-(4,5-dihydro-1H-imidazol-2-yl)pyrazine
   2-(4,5-dihydroimidazol-2-yl)quinoline
   2-(4-carbamimidoylphenyl)-1H-indole-6-carboxamidine
   2-(4-chlorophenoxy)acetamidine
   2-(4-pyridyl)cycloformanizine
   2,2-dimethylpropionamidine
   2,4-dimethyl-2-imidazoline
   2,4-dioxo-1-(4-pyridinylmethyl)hexahydro-5-pyrimidinecarboximidamide
   2-amidinopyridine
   2-amidinopyrimidine
   2-amidinothiophene
   2-amino-4(3H)-pyrimidone
   2-amino-4-[2-(3-amino-4-carbamimidoylphenyl)vinyl]benzamidine
   2-benzyl-2-imidazoline
   2-carbamimidoylacetamide
   2-cyclopropylacetamidine
   2-ethyl-4-methylimidazoline
   2-furancarboximidamide
   2-imino-4,6-dimethylpiperidine
   2-imino-4-methylpiperidine
   2-iminopiperidine
   2-methyl-1,4,5,6-tetrahydropyrimidine-4-carboxylic acid, sodium salt
   2-methyl-2-imidazoline
   2-methylthiazole-4-carboxamidine
   2-n-propyl-2-imidazoline
   2-phenoxyacetamidine
   2-phenyl-2-imidazoline
   2-phenylethanimidamide
   2-thiophenecarboxamidine
   3-(4,5-dihydro-1H-imidazol-2-yl)phenylamine
   3-(aminoiminomethyl)benzoic acid, sodium salt
   3-[2-(1-methyl-1,4,5,6-tetrahydropyrimidin-2-yl)-vinyl]phenol
   3-amino-1H-isoindole
   ethyl 3-amino-3-iminopropanoate
   3-aminobenzamidine
   3-aminomethylbenzamidine
   3-butenamidine
   3-chlorobenzamidine
   3-chloropropanimidamide
   3-furancarboximidamide
   3-methylbenzenecarboximidamide
   3-nitrobenzenecarboximidamide
   3-thiophenecarboximidamide
   4-(1-pyrrolidine)butyramidine
   4′,6-diamidino-2-phenylindole
   4-[5-(4-carbamimidoylphenoxy)pentyloxy]benzamidine
   4-amidinoanisole
   4-amidinobenzamide
   4-amidinobenzoic acid, sodium salt
   4-amidinopyridine
   4-aminobenzamidine
   4-aminomethylbenzamidine
   4-anisamidine
   4-chlorobenzamidine
   4-chlorobenzene-1-carboximidamide
   4-hydroxybenzamidine
   4-methoxycarbonylbenzamidine
   4-methylbenzamidine
   4-pyridinecarboxamidine
   5,5-dimethyl-2-phenyl-4,5-dihydro-1H-imidazole
   6H-6-imino-(2,3,4,5-tetrahydropyrimido)[1,2-c]-[1,3]benzothiazine
   amidinophenylpyruvic acid, sodium salt
   aminoacetamidine dihydrobromide
   benzamidine
   benzyl(4,5-dihydro-1H-imidazol-2-ylmethyl)phenylamine
   chloroacetamidine
   cyclobutanecarboxamidine
   cyclohexanecarboxamidine
   cyclopentanecarboximidamide
   cyclopropanecarboxamidine
   cyclopropylcarbamidine
   deoxypeganine
   ethyl amidinoacetate
   heptanimidamide
   N-(3,4-dihydro-2H-pyrrol-5-yl)-N-phenylamine
   N-(3-chlorophenyl)acetamidine
   N,N′-bis[3-(4,5-dihydro-1H-imidazol-2-yl)phenyl]urea dipropanoate
   N,N′-diphenylbenzamidine
   N,N′-di-p-tolylacetamidine
   N-phenylbenzamidine
   p-aminobenzamidine
   p-aminobenzamidine HCl
   pyrazine-2-carboxamidine
   tert-butylcarbamidine
   tolazoline, and
   trifluoroacetamidine
(b) In some embodiments, the amidine-type imines may be chosen from, for example:
   1,2-dimethyl-1,4,5,6-tetrahydropyrimidine
   1-methyl-2-(2-thiophen-2-ylvinyl)-1,4,5,6-tetrahydro-pyrimidine
   2,3,4,6,7,8,9,10-octahydropyrimido[1,2-a]azepine
   2,3,4,6,7,8-hexahydropyrrolo[1,2-a]pyrimidine
   2-methylpropanimidamide
   2-methylpropionamidine
   3-amidinopyridine
   3-pyridinecarboxamidine
   6-(dibutylamino)-1,8-diazabicyclo[5.4.0]undec-7-ene
   acetamidine
   butyramidine, and
   propionamidine.

In the compositions intended for hair relaxing, decurling or straightening processes, the imine not belonging to the hydroxide family may be present in a molar concentration ranging from 0.1M to 2M, which corresponds to concentrations of from 0.7% to 80% by weight relative to the total weight of the composition, such as in concentrations of from 0.2M to 1M, which corresponds to concentrations of from 1.4% to 40% by weight relative to the total weight of the composition.

The pH of the compositions may range from 9.6 to 14, such as from 11 to 13.

In some embodiments, the imine not belonging to the hydroxide family is the only relaxing active agent in the composition.

The compositions may also comprise at least one known reducing agent, such as thioglycolic acid or thiolactic acid and ester and amide derivatives thereof, for example glyceryl monothioglycolate, cysteamine and its C₁-C₄ acyl derivatives such as N-acetylcysteamine, N-propionylcysteamine, cysteine, N-acetylcysteine, thiomalic acid, pantethine, 2,3-dimercaptosuccinic acid, sulfites and bisulfites of an alkali metal or alkaline-earth metal, the N-(mercaptoalkyl)-ω-hydroxyalkylamides described in Patent Application EP-A-354,835, the N-mono- or N,N-dialkylmercapto-4-butyramides described in Patent Application EP-A-368,763, the aminomercaptoalkylamides described in Patent Application EP-A-432,000, the N-(mercaptoalkyl)succinamic acid and N-(mercaptoalkyl)succinimide derivatives described in Patent Application EP-A-465,342, the alkylamino mercaptoalkylamides described in Patent Application EP-A-514,282, the azeotropic mixture of 2-hydroxypropyl thioglycolate and of (2-hydroxy-1-methyl)ethyl thioglycolate described in Patent Application FR-A-2 679,448, the mercaptoalkylaminoamides described in Patent Application FR-A-2,692,481, the N-mercaptoalkylalkanediamides described in Patent Application EP-A-653,202 and the formamidinesulfinic acid derivatives described in Patent Publication No. WO 02/39965.

When the compositions comprise at least one reducing agent, the agent is present in a maximum concentration of 20% by weight, for example, from 0.1% to 10% by weight relative to the total weight of the composition.

The compositions may also contain known hydroxides chosen from alkali metal or alkaline-earth metal or transition metal or organic hydroxides such as sodium hydroxide, potassium hydroxide, lithium hydroxide, rubidium hydroxide, cesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminum hydroxide, guanidinium hydroxide, and quaternary ammonium hydroxides.

When the compositions comprise at least one hydroxide, this hydroxide may be present in a concentration ranging from 0.01% to 3.5% by weight, such as from 0.05% to 1.5% by weight relative to the total weight of the composition.

In some embodiments, the compositions contain 0% of base belonging to the hydroxide family such as alkali metal, alkaline-earth metal, transition metal, or organic hydroxides.

In some embodiments, the compositions comprise from 0 to 50% water, such as from 0 to 30% or even from 0 to 20% of water.

According to certain embodiments, the basic compositions also comprise at least one surfactant chosen from nonionic, anionic, cationic and amphoteric surfactants, such as alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and other hydroxypropyl ether nonionic surfactants.

When the basic compositions comprise at least one surfactant, the surfactant may be present in a maximum concentration of 30% by weight, such as from 0.5% to 10% by weight relative to the total weight of the composition.

The basic compositions may also comprise at least one treating agent of cationic, anionic, nonionic or amphoteric nature to improve the cosmetic properties of the hair or to attenuate or avoid its degradation.

Suitable treating agents include, but are not limited to, those described in French Patent Nos. 2,598,613 and 2,470,596. It is also possible to use as treating agents volatile or non-volatile, linear or cyclic silicones and mixtures thereof, polydimethylsiloxanes, quaternized polyorganosiloxanes such as those described in French Patent Application No. 2,535,730, polyorganosiloxanes containing aminoalkyl groups modified with alkoxycarbonylalkyl groups, such as those described in U.S. Pat. No. 4,749,732, polyorganosiloxanes such as the polyoxyalkyl polydimethylsiloxane copolymer of the dimethicone copolyol type, a polydimethylsiloxane containing stearoxy end groups (stearoxy dimethicone), a dialkylammonium acetate polydimethylsiloxane or a polydimethylsiloxane polyalkylbetaine copolymer described in British Patent No. 2,197,352, polysiloxanes organomodified with mercapto or mercaptoalkyl groups, such as those described in French Patent No.1,530,369 and in European patent application EP 295,780, and also silanes such as stearoxytrimethylsilane.

The basic compositions may also comprise other treating ingredients such as cationic polymers, for example, those used in the compositions of French Patent Nos. 79/32078 (2,472,382) and 80/26421 (2,495,931); ionene cationic polymers, such as those used in the compositions of Luxembourg Patent No. 83,703; basic amino acids such as lysine or arginine; acidic amino acids (such as glutamic acid or aspartic acid) and peptides and derivatives thereof; protein hydrolysates; waxes; swelling agents; penetrating agents or agents for reinforcing the efficacy of the reducing agent, such as a SiO₂/PDMS (polydimethylsiloxane) mixture, dimethylisosorbitol, urea and its derivatives, pyrrolidone, N-alkylpyrrolidones, thiamorpholinone, alkylene glycol or dialkylene glycol alkyl ethers, for example propylene glycol monomethyl ether, dipropylene glycol monomethyl ether, ethylene glycol monoethyl ether and diethylene glycol monoethyl ether, and 2-imidazolidinone; other compounds such as fatty alcohols and lanolin derivatives; active ingredients such as pantothenic acid; agents for preventing hair loss; antidandruff agents; thickeners; suspending agents; sequestering or complexing agents; opacifiers; sunscreens; fragrances; and preserving agents.

In some embodiments, the compositions comprise at least one imine not belonging to the hydroxide family that relaxes keratin fibers without being placed in contact beforehand with an organic solvent.

The compositions may be in the form of a thickened cream so as to hold the hair as stiff as possible. These creams are made in the form of “heavy” emulsions, for example based on glyceryl stearate, glycol stearate, self-emulsifying waxes, or fatty alcohols.

Liquids or gels containing thickeners, such as carboxyvinyl polymers or copolymers that “stick” the hairs together and hold them in a smooth position during the leave-in time, may also be used.

The compositions may also comprise at least one adjuvant chosen from silicones in soluble, dispersed or microdispersed form; nonionic, anionic, cationic and amphoteric surfactants; ceramides, glycoceramides and pseudoceramides; vitamins and provitamins including panthenol; plant, animal, mineral and synthetic oils; waxes other than ceramides, glycoceramides, and pseudoceramides; water-soluble and liposoluble, silicone-based or non-silicone-based sunscreens; nacreous agents and opacifiers; sequestering agents; plasticizers; solubilizers such as lower alcohols, e.g., ethanol, propanol, and isopropanol; acidifying agents; mineral and organic thickeners; antioxidants; hydroxy acids; penetrating agents; fragrances; and preserving agents.

The present disclosure also relates to kits comprising at least two compartments, one of the compartments (i) comprising, in a cosmetically acceptable medium free of polyhydroxylated alkane, at least one imine not belonging to the hydroxide family, which is capable of reacting with the cystines of keratin fibers, via a beta-elimination reaction producing dehydroalanine and leading to the formation of lanthionine, to relax keratin fibers in less than 60 minutes.

The kits may also comprise an additional composition (ii) for caring for, conditioning, making up, removing makeup from, protecting, cleansing, or washing keratin fibers.

The compositions of the kits are packaged in separate compartments, containers or devices, optionally accompanied by suitable, identical or different application means, such as fine brushes, coarse brushes, and sponges.

The disclosure also provides processes for relaxing keratin fibers using a cosmetic composition comprising, in a cosmetically acceptable medium free of polyhydroxylated alkane, at least one imine not belonging to the hydroxide family, which is capable of reacting with the cystines of the keratin fibers, via a beta-elimination reaction to produce dehydroalanine and lead to the formation of lanthionine, to relax the keratin fibers in less than 60 minutes.

The relaxing time may be less than 40 minutes, or even less than 30 minutes.

In the hair relaxing or decurling or straightening processes described herein, the basic composition is applied to the hair and the hair is then subjected to mechanical reshaping, giving it a new shape, via an operation of smoothing-out the hair with a large-toothed comb, with the back of a comb or by hand. After a leave-in time of from 5 to 60 minutes, for example, from 5 to 40 minutes, the hair is smoothed out again and is then rinsed thoroughly.

According to the disclosure, after applying the compositions described herein, the head of hair may be subjected to a heat treatment by heating to a temperature from 30 to 60° C. In practice, this operation may be performed using a hairstyling hood, a hairdryer, an infrared ray dispenser, and other standard heating devices.

It is also possible to use, as a means of both heating and smoothing out the hair, a hot iron at a temperature from 60 to 220° C., for example, from 120 to 200° C.

The disclosure also relates to the use of an imine not belonging to the hydroxide family as an active agent for relaxing keratin fibers.

The disclosure also relates to an active agent for relaxing keratin fibers, by means of a beta-elimination reaction producing dehydroalanine and leading to the formation of lanthionine, comprising at least one imine not belonging to the hydroxide family.

The disclosure may be understood more clearly with the aid of the non-limiting examples that follow. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE 1

A simplified relaxing composition was prepared, containing, as relaxing active agent, 1,1-dimethylbiguanide (Registry Number [1115-70-4]) at a concentration of 0.75M in water. The pH of the composition was 13.6. The composition was applied to naturally curly/frizzy African hair for 15 minutes at a temperature of 30° C. The hair was efficiently relaxed, easy to comb and style, and felt soft.

### EXAMPLE 2

A simplified relaxing composition was prepared, containing, as relaxing active agent, 1,1-dimethylbiguanide (Registry Number [1115-70-4]) at a concentration of 0.5M in water. The pH of the composition was 13.5. This composition was applied to naturally curly/frizzy African hair for 25 minutes at a temperature of 30° C. The hair was efficiently relaxed, easy to comb and style, and felt soft.

### EXAMPLE 3

A simplified relaxing composition was prepared, containing as relaxing active agent, 1,3,4,6,7,8-hexahydro-2H-pyrimido[1,2-a]pyrimidine (Registry Number [5807-14-7]) at a concentration of 0.7M in water. The pH of the composition was 13.5. This composition was applied to naturally curly/frizzy African hair for 15 minutes at a temperature of 30° C. The hair was efficiently relaxed, easy to comb and style, and felt soft.

### EXAMPLE 4

A simplified relaxing composition was prepared, containing as relaxing active agent, 1,3,4,6,7,8-hexahydro-2H-pyrimido[1,2-a]pyrimidine (Registry Number [5807-14-7]) at a concentration of 0.5M in water. The pH of the composition was 13.4. This composition was applied to naturally curly/frizzy African hair for 25 minutes at a temperature of 30° C. The hair was efficiently relaxed, easy to comb and style, and felt soft.

1. A cosmetic composition for relaxing keratin fibers comprising, in a cosmetically acceptable medium free of polyhydroxylated alkane, at least one imine not belonging to the hydroxide family, the cosmetically acceptable medium and the at least one imine being chosen such that the at least one imine is reactive with the cystines of the keratin fibers via a beta-elimination reaction to produce dehydroalanine and lead to formation of lanthionine to relax the keratin fibers in less than 60 minutes.

2. The composition according to claim 1, wherein the relaxation time is less than 40 minutes.

3. The composition according to claim 2, wherein the relaxation time is less than 30 minutes.

4. The composition according claim 1, wherein the at least one imine is present in a concentration ranging from 0.1 to 2 M.

5. The composition according claim 4, wherein the at least one imine is present in a concentration ranging from 0.2 to 1 M.

6. The composition according to claim 1, wherein in the pH ranges from 9.6 to 14.

7. The composition according to claim 6, wherein in the pH ranges from 11 to 13.

8. The composition according to claim 1, wherein the composition comprises 0% of base belonging to the hydroxide family.

9. The composition according to claim 1, wherein the composition comprises from 0 to 50% water.

10. The composition according to claim 9, wherein the composition comprises from 0 to 30% water.

11. The composition according to claim 10, wherein the composition comprises from 0 to 20% water.

12. The composition according to claim 1, wherein the at least one imine is chosen from guanidine and amidine imines.

13. The composition according to claim 12, wherein the guanidine imines are chosen from:
N,N-dimethylguanidine,
N,N′,N″-trimethylguanidine,
N-methylguanidine,
N,N-diethylguanidine,
N-ethylguanidine,
N,N-dimethylimidodicarbonimidic diamide,
N-(tert-butyl)-N″-methylguanidine,
N-(4-aminobutyl)guanidine,
1,3,4,6,7,8-hexahydro-2H-pyrimido[1,2-a]pyrimidine,
N,N″-diisopropylguanidine,
1-methyl-1,3,4,6,7,8-hexahydro-2H-pyrimido[1,2-a]pyrimidine,
N″-(tert-butyl)-N,N,N′,N′-tetramethylguanidine,
N,N,N′,N′-tetraethylguanidine,
N,N″-di-tert-butylguanidine,
N″-[amino(imino)methyl]-N-phenylguanidine,
N,N′,N″-triisopropylguanidine,
pyrrolidine-1-carboximidamide,
N-phenylguanidine,
N-(tert-butyl)-N′,N″-dimethylguanidine,
N-(4-{[amino(imino)methyl]amino}butyl)guanidine,
N″-(tert-butyl)-N-methyl-N′-phenylguanidine,
N,N′,N″-tricyclohexylguanidine,
N-{3-[(4-{[amino(imino)methyl]amino}butyl)amino]propyl}guanidine,
N-(2-hydroxyethyl)-N-methylguanidine,
n-propylguanidine,
isopropylguanidine,
(2-methylpropyl)guanidine,
(2-ethylhexyl)guanidine,
(2-hydroxyethyl)guanidine,
(2-methoxyethyl)guanidine,
o-, m- or p-hydroxybenzylguanidine,
cyclopentylguanidine,
cyclohexylguanidine,
cyclohexylmethylguanidine,
4-oxacyclohexylguanidine,
glucopyranosylguanidine,
biguanido(guanylguanidine),
1-methylbiguanide,
1-ethylbiguanide,
1-n-propylbiguanide,
1-isopropylbiguanide,
1-(2-methylpropyl)biguanide,
1-(2-hydroxyethyl)biguanide,
1-phenylbiguanide,
1-benzylbiguanide,
1-cyclopentylbiguanide,
1-cyclohexylbiguanide,
1,1-dimethylbiguanide,
1,1-diethylbiguanide,
1-ethyl-1-methylbiguanide,
1-methyl-1-ethylguanidine,
1-methyl-1-n-propylguanidine,
1-methyl-1-isopropylguanidine,
1,1-bis(2-hydroxyethyl)guanidine,
1-methyl-1-phenylguanidine,
3-methyl-1-ethylguanidine,
3-methyl-1-n-propylguanidine,
3-methyl-1-isopropylguanidine,
1,3-bis(2-hydroxyethyl)guanidine,
1,3-dibenzylguanidine,
3-methyl-1-benzylguanidine,
1,3-dicyclopentylguanidine,
3-methyl-1-cyclopentylguanidine,
3-methyl-1-cyclohexylguanidine,
1,3-di(4-oxacyclohexyl)guanidine,
3-methyl-1-(4-oxacyclohexyl)guanidine,
3-methyl-1-(glucopyranosyl)guanidine, and
N-[bis(dimethylamino)methylene]-N′,N″-diisopropylguanidine.

14. The composition according to claim 12, wherein the amidine imines are chosen from:
1,2-dimethyl-1,4,5,6-tetrahydropyrimidine,
1-methyl-2-(2-thiophen-2-ylvinyl)-1,4,5,6-tetrahydropyrimidine,
2,3,4,6,7,8,9,10-octahydropyrimido[1,2-a]azepine,
2,3,4,6,7,8-hexahydropyrrolo[1,2-a]pyrimidine,
2-methylpropanimidamide,
2-methylpropionamidine,
3-amidinopyridine,
3-pyridinecarboxamidine,
6-(dibutylamino)-1,8-diazobicyclo[5.4.0]undec-7-ene,
acetamidine,
butyramidine, and
propionamidine.

15. The composition according claim 1, further comprising at least one adjuvant chosen from silicones in soluble, dispersed or microdispersed form; nonionic, anionic, cationic and amphoteric surfactants; ceramides; glycoceramides; pseudoceramides; vitamins and provitamins; plant, animal, mineral and synthetic oils; waxes other than ceramides, glycoceramides and pseudoceramides; water-soluble and liposoluble, silicone-based or non-silicone-based sunscreens; nacreous agents; opacifiers; sequestering agents; plasticizers; solubilizers; acidifying agents; mineral and organic thickeners; antioxidants; hydroxy acids; penetrating agents; fragrances; and preserving agents.

16. The composition according claim 14, wherein the vitamins and provitamins are chosen from panthenol.

17. A kit for relaxing keratin fibers comprising at least two compartments, wherein one of the compartments comprises a first composition comprising, in a cosmetically acceptable medium free of polyhydroxylated alkane, at least one imine not belonging to the hydroxide family, the cosmetically acceptable medium and the at least one imine being chosen such that the at least one imine is reactive with the cystines of the keratin fibers, via a beta-elimination reaction to produce dehydroalanine and lead to a formation of lanthionine, to relax the keratin fibers in less than 60 minutes.

18. The kit according to claim 17, further comprising a second composition for caring for, conditioning, making up, removing makeup from, protecting, cleansing, or washing keratin fibers.

19. A process for relaxing keratin fibers comprising:
applying to the keratin fibers a cosmetic composition comprising, in a cosmetically acceptable medium free of polyhydroxylated alkane, at least one imine not belonging to the hydroxide family,
wherein the at least one imine reacts with the cystines of the keratin fibers, via a beta-elimination reaction to produce dehydroalanine and lead to the formation of lanthionine, to relax the keratin fibers in less than 60 minutes.

20. The process for relaxing keratin fibers according to claim 19, further comprising subjecting the keratin fibers to a heat treatment by heating the keratin fibers to a temperature ranging from 30 to 60° C.

21. The process for relaxing keratin fibers according to claim 19, further comprising heating and smoothing out the keratin fibers with a hot iron at a temperature of ranging from 60 to 220° C.

22. The process for relaxing keratin fibers according to claim 21, wherein the iron temperature ranges from 120 to 200° C.

23. The process for relaxing keratin fibers according to claim 19, wherein the keratin fibers are relaxed in less than 40 minutes.

24. The process for relaxing keratin fibers according to claim 23, wherein the keratin fibers are relaxed in less than 30 minutes.
